(19) United States
(12) Patent Application Publication
(10) Pub. No.: US 2021/0313031 A1
(43) Pub. Date: Oct. 7, 2021

(54) SYSTEM, METHODS, AND APPARATUS FOR REMOTE VERIFICATION OF PHARMACY PRESCRIPTION PREPARATION

(71) Applicant: INNOVATION ASSOCIATES, INC., Johnson City, NY (US)

(72) Inventors: Phil Samples, Johnson City, NY (US); Marla Ocker, Johnson City, NY (US); Chris Cole, Johnson City, NY (US)

(21) Appl. No.: 16/891,685

(22) Filed: Jun. 3, 2020

Related U.S. Application Data
(60) Provisional application No. 63/004,885, filed on Apr. 3, 2020.

(51) Int. Cl. G16H 20/10; G16H 40/67; G16H 10/60; G16H 30/00; G16H 40/20

(52) U.S. Cl. CPC G16H 20/10; G16H 40/67; G16H 40/20; G16H 30/00; G16H 10/60

(57) ABSTRACT

A system, method, and apparatus for pharmacy remote verification is disclosed. In an example, a pharmacy remote verification apparatus is configured to receive, from a local pharmacy system, a set of prescription fill information for remote verification for a plurality of prescriptions. The pharmacy remote verification apparatus creates an ordered list of the prescriptions based on an urgency such that a most urgent prescription is ordered first for review. The pharmacy remote verification apparatus receives, from a remote verifier device, a request message to review a prescription. In response, the pharmacy remote verification apparatus causes the first ordered prescription to be displayed on the remote verifier device. The remote verifier device may provide a verified message, which is relayed by the pharmacy remote verification apparatus to the local pharmacy system to enable the corresponding prescribed medication to be released to a patient.

### PRIORITY CLAIM

This application claims priority to and the benefit of U.S. Provisional Patent Application Ser. No. 63/004,885, filed on Apr. 3, 2020, the entire disclosure of which is hereby incorporated by reference.

### BACKGROUND

Healthcare in the United States is at a pivotal point where available resources fail to meet patient demand. Oftentimes, the unavailability of the healthcare system to meet patient demand results in suboptimal care and increased costs. The prescription fulfillment industry is not immune from these nationwide healthcare system trends. Currently, the prescription fulfillment industry is being consolidated into large multinational corporations that have numerous retail locations with prescription refill capability. To reduce costs, each retail location may have only one or two resident pharmacists, with the remainder of the pharmacy staff being limited to pharmacy technicians or unlicensed assistants.

Each pharmacist is tasked with providing patient consultations, administering immunizations, performing prescription compatibility checks, and refilling prescriptions. In many situations, a pharmacy technician (i.e., a Certified Pharmacy Technician) may refill prescriptions when the pharmacist is performing other tasks or is otherwise not available. However, the pharmacist still has to verify the filled prescription before the prescribed medication can be released to a patient. To verify a prescription, a pharmacist ensures the medication in a medication container matches information in a prescription or medication order, including medication name/pill marking, dosage, pill color, quantity, etc. The pharmacist also ensures the patient information printed on the container matches the patient information provided with the prescription including name, age, birthdate, gender, medical condition/disease, etc.

To help provide verification when a pharmacist is not available, some pharmacy systems use point-to-point remote verification. In these known systems, a designated remote pharmacist reviews filled prescriptions. This includes reviewing video or photos of the prescription preparation process, images of pills on a counting tray/in the medication container, and/or images of a label on the medical container. An issue with these known systems is that only one or a few remote pharmacists have access to the local system for verification. In addition, remote verification requires specialized software on a remote computer system, and usually only occurs over a live connection with the local pharmacy. If the remote and local pharmacists are both unavailable or occupied with other tasks, prescriptions may not be timely verified for patient pickup. As such, current pharmacy systems may be inadequate in providing timely prescription fills for patients.

### SUMMARY

Example systems, methods, and apparatus are disclosed herein for a remote pharmacy verification system. The example systems, methods, and apparatus provide enable prescription fill information to be created and entered at local pharmacies. The prescription fill information is transmitted to a centralized remote verification server, which arranges each prescription based on an urgency. The systems, methods, and apparatus permit configuration such that local pharmacies of a same pharmacy provider are arranged together such that the most urgent prescriptions of the group are reviewed first. The systems, methods, and apparatus may order the prescriptions using common urgency rules of the local pharmacies. Additionally, if there are differences between urgency rules, the systems, methods, and apparatus combine and/or normalize the rules to provide a review arrangement that is consistent with each local pharmacy's queuing structure.

In some embodiments, the local pharmacy systems transmit prescription fill information to the remote verification server after a remote verification feature is enabled. For example, a pharmacy technician may enable remote verification when a local pharmacist is unavailable. Additionally or alternatively, the local pharmacy system may automatically transmit prescription fill information to the remote verification server after a prescription queue reaches a threshold, such as five, ten, twenty, etc. prescriptions.

The remote verification server provided by the systems, methods, and apparatus hosts a website or application interface that enables remote verifier devices to access the prescription fill information. The server selects which prescription is provided for review based on the arranged urgency. This configuration ensures that the most urgent prescriptions are reviewed first by any of the connected remote verifier devices.

After review, a remote verifier device provides a message that is indicative as to whether a prescription is successfully verified or cannot be verified by the reviewer. For instance, a reviewer cannot verify prescription fill information if at least some of the information is missing or incorrect, such as a patient name, medication/pill name, medication dosage, pill count, etc. If a prescription is indicated as being verified, the remote verification server transmits a message to the local pharmacy indicative of the successful verification. The message may be used by the local pharmacy to unlock a compartment of a cabinet to enable a medical container associated with the verified prescription to be removed and conveyed to a patient. In other instances, the message may cause a local pharmacy computer system to change an indicator light on the compartment to a color, code, text, etc. that is indicative that the medication container can be removed. If a prescription cannot be successfully verified, the remote reviewer provides at least one reason for the unsuccessful verification, which is routed by the remote verification server to the local pharmacy. A technician or local pharmacist may then take steps to rectify the prescription fill error, and cause the revised prescription fill information to be transmitted to the remote verifier device of the reviewer.

In contrast to known pharmacy systems, the disclosed remote verification server enables remote verifier devices to access prescription fill information without the need for specialized software. Instead, the remote verification server provides a web-interface or an application programmable interface (“API”) for transmission of the prescription fill information (and receiving verification information). Without the need for specialized software, a greater number of remote verifier devices may connect to the remote verification server, as long as the reviewing user is a registered/authenticated user. In some embodiments, a remote reviewer may use their smartphone or tablet computer to review prescription fill information, thereby enabling mobile verification.

Also in contrast to known systems, the verification server enables prescription fill information to be reviewed when remote reviewers are available. A live connection between a remote reviewer and a local pharmacy is not needed. As such, a pharmacy system can assign remote reviewers around the world (or large geographic regions) to provide for around-the-clock prescription verification. Altogether, the above systems, methods, and apparatus disclosed herein reduce prescription verification queue times to ensure that patients receive their prescribed medications in a timely manner with minimal waiting.

In light of the disclosure herein and without limiting the disclosure in any way, in a first aspect of the present disclosure, which may be combined with any other aspect listed herein unless specified otherwise, a pharmacy remote verification system includes a local pharmacy database configured to store prescription fill information for a plurality of prescriptions and a local pharmacy processor communicatively coupled to the local pharmacy database and configured to receive the prescription fill information for storage in the local pharmacy database. The local pharmacy processor is configured to transmit a set of the prescription fill information for remote verification contingent upon at least one of receiving an indication of a remote verification mode being enabled, or the set of the prescription fill information including images of a prescription preparation process. The pharmacy remote verification system also includes a remote pharmacy database configured to store the set of the prescription fill information and a remote verification server communicatively coupled to the remote pharmacy database and communicatively coupled to the local pharmacy processor via a network connection. The remote verification server includes at least one interface configured to provide a remote verifier device access for verification of the set of the prescription fill information. The remote verification server is configured to arrange prescriptions of the set of the prescription fill information based on an urgency of the prescriptions such that a most urgent prescription is selected for review. After receiving a request to review a prescription preparation, the remote verification server causes a most urgent, un-reviewed prescription including the corresponding prescription fill information to be displayed on the remote verifier device. The remote verification server is configured to receive a verified message or an unverified message from the remote verifier device. If the verified message is received, the remote verification server is configured to store a verified indication to the prescription fill information of the verified prescription and transmit a verified message to the local pharmacy processor causing the local pharmacy processor to indicate the respective prescription is verified and available to convey to an associated patient. If the unverified message is received, the remote verification server is configured to store an unverified indication to the prescription fill information of the unverified prescription and transmit an unverified message to the local pharmacy processor causing the local pharmacy processor to indicate at least one reason why the prescription cannot be verified, thereby preventing the respective prescription from being made available to the associated patient, and enabling the at least one reason to be corrected.

In accordance with a second aspect of the present disclosure, which may be used in combination with any other aspect listed herein unless stated otherwise, the prescription fill information includes at least one of a patient identifier, a medication identifier, a medication quantity identifier, a dosage identifier, an urgency identifier, a weight value, a medication shape identifier, a medication manufacturer indicator, a dosage form indicator, a color indicator, a marking indicator, a DEA code, an expiration date, medication directions, or at least one image of the prescription preparation process.

In accordance with a third aspect of the present disclosure, which may be used in combination with any other aspect listed herein unless stated otherwise, the at least one image includes an image of medication in a medication container, an image of a medication container label, an image of a pill counter display, an image of a weight scale display, or an image of the medication showing a dosage or a name of the medication.

In accordance with a fourth aspect of the present disclosure, which may be used in combination with any other aspect listed herein unless stated otherwise, the at least one reason why the prescription cannot be verified includes at least one of an incorrect medication entry, an incorrect medication strength entry, an incorrect medication quantity entry, an incorrect directions entry, an incorrect patient data entry, an incorrect medication fill, an incorrect medication strength fill, an incorrect medication quantity fill, a dispenser over-count fill, or poor quality of at least one image.

In accordance with a fifth aspect of the present disclosure, which may be used in combination with any other aspect listed herein unless stated otherwise, the remote verification server is configured to transmit a reviewing message to the local pharmacy processor when the prescription fill information of the most urgent prescription related the local pharmacy processor is provided for verification to the remote verifier device.

In accordance with a sixth aspect of the present disclosure, which may be used in combination with any other aspect listed herein unless stated otherwise, the local pharmacy processor is configured to transmit the set of the prescription fill information to the remote verification server if a number of prescriptions needing verification stored in the local pharmacy database exceeds a threshold.

In accordance with a seventh aspect of the present disclosure, which may be used in combination with any other aspect listed herein unless stated otherwise, the urgency includes at least one of a fill next indicator, an urgent importance indicator, a high importance indicator, a standard importance indicator, or a fill by date/time indicator.

In accordance with an eighth aspect of the present disclosure, which may be used in combination with any other aspect listed herein unless stated otherwise, the remote verification server is configured to determine a current date/time equals or is about to approach the date/time indicator of an unverified prescription of the set of the prescription fill information, and change the urgency of the determined unverified prescription to at least one of the fill next indicator or the urgent importance indicator.

In accordance with a ninth aspect of the present disclosure, which may be used in combination with any other aspect listed herein unless stated otherwise, the local pharmacy processor is configured to receive an indication that the remote verification mode is terminated, and transmit a termination message that is indicative of the remote verification mode termination to the remote verification server.

In accordance with a tenth aspect of the present disclosure, which may be used in combination with any other aspect listed herein unless stated otherwise, the remote verification server is configured to receive the termination message and remove, from the remote pharmacy database, prescriptions of the set of the prescription fill information that have not been reviewed.

In accordance with an eleventh aspect of the present disclosure, which may be used in combination with any other aspect listed herein unless stated otherwise, the local pharmacy processor is communicatively coupled to the remote verification server via at least one of a private network connection or secure Internet connection, and the remote verification server is communicatively coupled to the remote verifier device via a public network connection.

In accordance with a twelfth aspect of the present disclosure, which may be used in combination with any other aspect listed herein unless stated otherwise, the local pharmacy processor is configured to, after receiving the verified message for the respective prescription, cause a medication cabinet to at least one of display a visual indication at a compartment of the cabinet that includes a medication container related to the verified prescription, the visual indication being indicative that the medication container can be conveyed to the patient, or unlock the compartment of the cabinet that includes the medical container for the verified prescription.

In accordance with a thirteenth aspect of the present disclosure, which may be used in combination with any other aspect listed herein unless stated otherwise, a pharmacy remote verification apparatus stores non-transitory computer-readable instructions, which when executed, cause the apparatus to receive, from a local pharmacy processor via a first network connection, a set of prescription fill information for remote verification for a plurality of prescriptions, create an ordered list of prescriptions of the set of the prescription fill information based on an urgency of the prescriptions such that a most urgent prescription is ordered first for review, receive, from a remote verifier device via a second network connection, a request message to review a prescription, after receiving the request message, cause the first ordered prescription including the related prescription fill information to be displayed on the remote verifier device, receive, from the remote verifier device, a verified message or an unverified message, if the verified message is received, store a verified indication to the prescription fill information of the verified prescription and transmit a verified message to the local pharmacy processor causing the local pharmacy processor to indicate the respective prescription is verified and available to convey to an associated patient, and if the unverified message is received, store an unverified indication to the prescription fill information of the unverified prescription and transmit a notification message to the local pharmacy processor causing the local pharmacy processor to indicate at least one reason why the respective prescription cannot be verified.

In accordance with a fourteenth aspect of the present disclosure, which may be used in combination with any other aspect listed herein unless stated otherwise, the pharmacy remote verification apparatus further includes stored non-transitory computer-readable instructions, which when executed, cause the apparatus to receive, from the local pharmacy processor, a correction message that is in response to the notification message, transmit at least some information from the correction message to the remote verifier device in relation to the unverified prescription; and receive, from the remote verifier device, the verified message or another unverified message.

In accordance with a fifteenth aspect of the present disclosure, which may be used in combination with any other aspect listed herein unless stated otherwise, the pharmacy remote verification apparatus further includes stored non-transitory computer-readable instructions, which when executed, cause the apparatus to remove the first ordered prescription from the ordered list, and transmit, to the local pharmacy processor, a verifying message indicative that the first ordered prescription is being reviewed.

In accordance with a sixteenth aspect of the present disclosure, which may be used in combination with any other aspect listed herein unless stated otherwise, the request message is received from a web browser of the remote verifier device, and the first ordered prescription, including the related prescription fill information, is formatted for display in the web browser of the remote verifier device.

In accordance with a seventeenth aspect of the present disclosure, which may be used in combination with any other aspect listed herein unless stated otherwise, the pharmacy remote verification apparatus further includes stored non-transitory computer-readable instructions, which when executed, cause the apparatus to receive a connection request message from the remote verifier device, transmit, to the remote verifier device, authentication prompt information, receive, from the remote verifier device, authentication information, and contingent upon the authentication information matching at least some authentication information stored in a database, transmit dashboard information that is indicative of a number of prescriptions that are included in within the ordered list of prescriptions.

In accordance with an eighteenth aspect of the present disclosure, which may be used in combination with any other aspect listed herein unless stated otherwise, the dashboard information includes a total number of connected local pharmacies, an indication of which of the connected local pharmacies have remote verification enabled, and a total number of prescriptions reviewed by a user of the remote verifier device.

In accordance with a nineteenth aspect of the present disclosure, which may be used in combination with any other aspect listed herein unless stated otherwise, the pharmacy remote verification apparatus further includes stored non-transitory computer-readable instructions, which when executed, cause the apparatus to determine at least one prescription of the set of prescription fill information does not include at least one image, and prevent the determined at least one prescription from being included in the ordered list of prescriptions.

In accordance with a twentieth aspect of the present disclosure, which may be used in combination with any other aspect listed herein unless stated otherwise, the set of prescription fill information for the plurality of prescriptions is a first set of prescription fill information for a first plurality of prescriptions, and the local pharmacy processor is a first local pharmacy processor. Additionally, the pharmacy remote verification apparatus further includes stored non-transitory computer-readable instructions, which when executed, cause the apparatus to receive, from a second local pharmacy processor via a third network connection, a second set of prescription fill information for remote verification for a second plurality of prescriptions, and create the ordered list of prescriptions for the first and second sets of the prescription fill information based on the urgency of the prescriptions such that the most urgent prescription is ordered first for review.

In a twenty-first aspect of the present disclosure, any of the structure, functionality, and alternatives disclosed in connection with any one or more of FIGS. 1 to 8 may be combined with any other structure, functionality, and alternatives disclosed in connection with any other one or more of FIGS. 1 to 8.

In light of the present disclosure and the above aspects, it is therefore an advantage of the present disclosure to provide a remote pharmacy verification system that enables a plurality of remote verifiers to review at any time prescription fill information provided by a plurality of local pharmacy systems.

It is another advantage of the present disclosure to provide remote verification without specialized software having to be installed at a device of a prescription reviewer.

It is a further advantage of the present disclosure to automatically route prescription fill information for remote verification after a prescription queue at a local pharmacy reaches a threshold.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a remote prescription verification system including a remote verification server and local pharmacy systems, according to an example embodiment of the present disclosure.

FIG. 2 is a flow diagram of an example procedure for creating and managing prescriptions at a local pharmacy system, according to an example embodiment of the present disclosure.

FIG. 3 is a diagram of the remote pharmacy server of FIG. 1, according to an example embodiment of the present disclosure.

FIG. 4 is a diagram of an example remote verifier dashboard that is provided by the remote verification server to a remote verifier device, according to an example embodiment of the present disclosure.

FIG. 5 is a diagram of a verification user interface (e.g., a webpage) provided by the remote verification server to a remote verifier device, according to an example embodiment of the present disclosure.

FIG. 6 is a diagram of a problem user interface (e.g., a webpage) provided by the remote verification server to a remote verifier device, according to an example embodiment of the present disclosure.

FIG. 7 is a flow diagram of an example procedure for verifying prescriptions using the remote verification server of FIGS. 1 and 3, according to an example embodiment of the present disclosure.

FIG. 8 is a flow diagram of an example procedure for verifying prescriptions using a two-step verification process using the remote verification server of FIGS. 1 and 3, according to an example embodiment of the present disclosure.

### DETAILED DESCRIPTION

Methods, systems, and apparatus are disclosed herein for remote pharmacy verification. As disclosed herein, remote pharmacy verification refers to a process for verifying a prescription for a medication without the verification procedure necessarily being carried out at the same physical location as the medication. Remote pharmacy verification is provided by a designated, certified individual reviewing or auditing digital prescription fill information. As disclosed herein, the prescription fill information includes at least one image recorded during the filling process to provide visual information that is indicative of medication preparation. In some instances, a local pharmacist may use the remote pharmacy system for verifying prescriptions at their own pharmacy in addition to other pharmacies.

Reference is made herein to (digital) prescription fill information. This information includes a patient identifier, a medication identifier, a medication quantity identifier, a dosage identifier, an urgency identifier, a weight value, a medication shape identifier, a medication manufacturer indicator, a dosage form indicator, a color indicator, a marking indicator, a Drug Enforcement Administration (“DEA”) code, an expiration date, medication directions, and/or at least one image of the prescription fill/preparation process. The image may include an image of medication (pills) in a medication container, an image of a medication container label, an image of a pill counter display, an image of a weight scale display, and/or an image of the medication showing a dosage or a name of the medication.

Reference is also made herein to prescriptions and medications. A prescription is generated by a local pharmacy based on a document (commonly referred to a medication order), which is provided by a clinician. A medication order designates a particular patient for receiving a specified dosage of a medication. References to a prescription herein refer to information from a medication order in addition to prescription fill information for a particular patient/medication. In other words, a prescription is a single medication fill event for a particular patient that is performed by a local pharmacy. A medication includes a pill, tablet, or other solid pharmaceutical drug dosage that is consumed by a patient. A medication may also include a compounded pharmaceutical that is prepared in a local pharmacy from two or more substances. While reference is made to pills/tablets, it should be appreciated that the remote verification system disclosed herein may be used for the preparation of any medication type.

A medication is dispensed to a patient in a medication container. Oftentimes, patient and/or medication information is printed by a local pharmacy on a label that is affixed to the medication container. In some instances, after medication preparation for remote verification, images are recorded of the medication dispensed within the container and/or the label affixed to the container.

Reference is also made to local pharmacy systems, local pharmacist (technician), and remote pharmacist. As disclosed herein, the local pharmacy system is configured for the preparation, filling, and dispensing of a prescribed medication to a patient. Medication filling/preparation is provided by a local pharmacist or technician. The example system, methods, and apparatus disclosed herein enable a remote pharmacist to review prescription fill information related to medication preparation/filling. The remote pharmacist is a remote verifier or reviewer that determines if the prescription was filled (and documented) correctly to enable the prescribed medication to be conveyed or dispensed to a patient. In some instances, a local pharmacist may provide remote or digital verification of prescriptions that originated from their own pharmacy such that the local pharmacist is also a remote pharmacist.

The example system, methods, and apparatus disclosed herein provide remote verification of prescriptions among pharmacists that are associated with the same company, region, pharmacy network, etc. For example, the system, methods, and apparatus prevent pharmacists employed by a first company from viewing or otherwise accessing prescriptions that are prepared within a pharmacy system of a second company. In another example, the methods, and apparatus disclosed herein prevent pharmacists designated in a first region by their employer from being able to view or otherwise access prescriptions that are prepared within a pharmacy system of a second region.

### I. REMOTE PHARMACY VERIFICATION EMBODIMENT

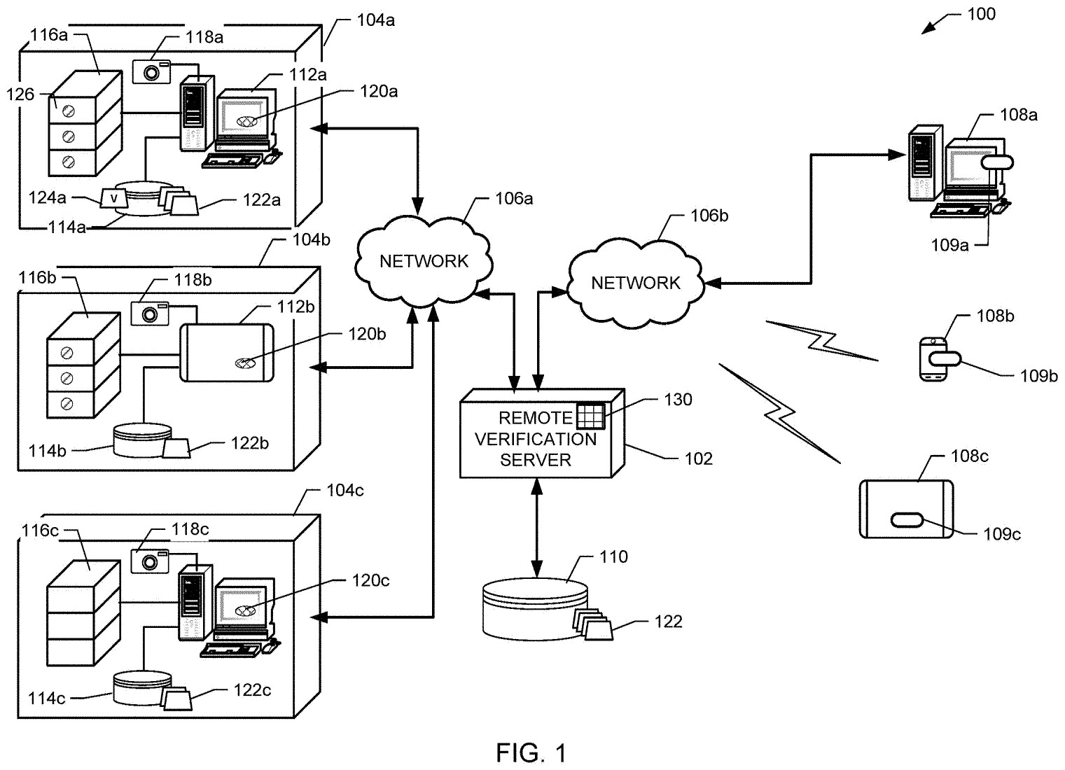

FIG. 1 is a diagram of a remote pharmacy verification system 100, according to an example embodiment of the present disclosure. The example system 100 includes a remote verification server 102 that is communicatively coupled to local pharmacy systems 104 via first network connections 106a. The remote verification server 102 is also communicatively coupled to remote verifier devices 108 via second network connections 106b. The remote verification server 102 is also communicatively coupled to a memory device 110.

In some embodiments, the first network connections 106a and the second network connections 106b may be part of the same network, such as a wide area network (e.g., the Internet), a cellular network, or combinations thereof. In other embodiments, the network connections 106a and 106b are part of different networks. For instance, the first network connections 106a may be part of a local area network (“LAN”) or other private network and the second network connections 106b may be a WAN or other public network.

The first network connections 106a may comprise individual connections to each of the local pharmacy systems 104a, 104b, and 104c. Each connection may comprise a virtual private area network, a virtual LAN, or other secure connection. The second network connections 106b may comprise individual connections to each of the remote verifier devices 108a, 108b, and 108c. Each connection may comprise a public connection, a virtual private area network, a virtual LAN, etc.

Each of the remote verifier devices 108 include a web browsing application 109 or other application for accessing the remote verification server 102. In an example, a web site hosted by the server 102 is displayed in the web browsing application 109 on the remote verifier devices 108. The website includes webpages that provide for remote verification. In some instances, the server 102 may provide one or more APIs for transmitting prescription fill information to the devices 108 and receiving response information from the devices 108.

In the illustrated embodiment, each of the local pharmacy systems 104 includes at least one local pharmacy computer or processor 112, a local memory device 114, a medication cabinet or bank 116, and a camera 118. The local pharmacy computer 112 includes, for example, a desktop computer, a laptop computer, a workstation, a smartphone, a tablet computer, a laptop, etc. The local pharmacy computer 112 may include one or more display devices and/or user input devices (e.g., a mouse, a keyboard, a touchscreen, etc.).

The local pharmacy computer 112 includes instructions stored in a memory device, which when executed by a processor of the computer 112, cause the computer 112 to execute a local pharmacy application 120. As described herein, the local pharmacy application 120 is configured to enable a local pharmacist or technician to enter or otherwise provide prescription fill information. The local pharmacy application 120 is also configured to operate with the remote verification server 102 for enabling remote verification of prescriptions.

The local pharmacy application 120 is configured to receive prescription fill information from one or more sources. In some embodiments, an electronic medication order may be received by the local pharmacy application 120 from a clinician computer (not shown). The electronic medication order may specify a patient name (and/or patient identifier), patient date of birth, medication order date, a medication name, a dosage, and/or a number of refills. The application 120 writes or stores the electronic medication order information as prescription fill information. In these instances, the application 120 may access a database (such as a database stored on the memory device 114a) of medication information to further supplement the medication order information. The database may store pill color information, pill shape information, pill marking information, pill size information, pill weight information, medication manufacturer information, etc. for each of the possible medications that can be prescribed to a patient. The application 120 identifies the medication information that matches the medication order and writes or stores the information as part of the prescription fill information.

In addition to above, the application 120 is configured to enable a local pharmacist to enter prescription fill information into the local pharmacy computer 112a. This may include any of the information mentioned above in addition to pill fill quantity, pill fill weight, patient insurance information, pill fill date/time, a prescription preparer identifier, patient health history, or other medications being consumed, etc.

Further, when enabled, the local pharmacy application 120 operates with the camera 118 to record at least one image of a pill preparation and/or fill process. The application 120 may store images, recorded by the camera 118, of pills placed in a counting tray, pills placed in a medication container, and/or any pill or compound preparation steps. The camera 118 may also record an image of a medication container label, a digital counter, and/or a weight scale. The recorded images are stored by the local pharmacy application 120 as part of the prescription fill information. The camera 118 may include any digital camera for recording still images, video, and/or audio.

The example local memory device 114 includes one or more databases for storing prescription fill information and verified prescription fill information. As shown in FIG. 1, the prescription fill information is designated as individual prescription records (e.g., prescriptions) 122. Verified prescriptions 124 are shown as being designated with a “V” character. For example, the local pharmacy system 104a includes a local memory device 114a with three prescriptions 122a awaiting review and one verified prescription 124a. The memory device 114 may include any database structure, such as SQL. Further, the memory device 114 may comprise any computer-readable medium, including random access memory (“RAM”), read only memory (“ROM”), flash memory, magnetic or optical disks, optical memory, or other storage media.

As shown in FIG. 1, each of the local pharmacy systems 104 includes a cabinet 116 with individual compartments or sections 126. After a medication has been filled, the container with the medication is stored in one of the compartments 126 of the cabinet 116. The medication container cannot be removed and conveyed to a patient until the related prescription fill information 122 has been verified by a pharmacist. In some embodiments, the cabinet 116 includes a lock for each compartment 126. The cabinet 116 and/or the local pharmacy computer 112 may not release the lock until a verified status is received for the corresponding prescription fill information. In other embodiments, each compartment 126 may include one or more indicators, which may include LED(s) or a display screen. An activation of a green LED indicator by the cabinet 116 or the local pharmacy computer 112 may be indicative that the related prescription fill information has been verified and the medication container may be removed for a patient. In contrast, activation of a red LED indicator by the cabinet 116 or the local pharmacy computer 112 may be indicative that the related prescription fill information has not yet been reviewed and the medication container may not be removed. Further, in some embodiments, activation of a flashing red LED indicator by the cabinet 116 or the local pharmacy computer 112 may be indicative that verification for the related prescription fill information has failed and that additional or corrected prescription fill information is needed. It should be noted that in some embodiments, the cabinet 116 may only provide for medication container storage, and not have any indicators or remotely controlled locks (as shown in the local pharmacy system 104c).

The example local pharmacy application 120 of FIG. 1 is configured to operate cooperatively with the remote verification server 102 for remote verification of prescription fill information. In the illustrated example, the remote verification server 102 includes a remote verification application 130. As described in more detail below, the remote verification application 130 provides an interface for the local pharmacy application 120. The interface may include one or more APIs that enable the local pharmacy application 120 to transmit prescriptions 122 for remote verification when a remote verification flag or feature is enabled at the local pharmacy system 104. The remote verification application 130 stores the received prescriptions 122 in the centralized memory device 110 until they can be transmitted for review via one of the remote verification devices 108. Each prescription 122 comprises prescription fill information and is stored as a separate entry or record with a unique identifier.

In some embodiments, after the applications 120 and 130 establish a connection with each other in response to a remove verification feature being activated, the memory devices 110 and 114 synchronize such that any prescriptions 122 awaiting verification in the local memory device 114 are copied to create corresponding records at the database 110. The remote copy of the prescription in the memory device 110 enables remote verification while the local, original version of the prescription in the local memory device 114 provides for localized management/tracking and/or availability for localized verification if a pharmacist is available.

The local pharmacy systems 104 may be part of one region or pharmacy network. Additionally, the remote verifier devices 108 are part of the same region or network. Prescriptions 122 from each of the local pharmacy systems 104 are aggregated and ordered by the remote verification server 102 for review by the devices 108. It should be appreciated that the server 102 may provide separate partitions for different pharmacy regions, companies, networks, etc. This compartmentation ensures that only pharmacists of the same network, region, etc. are able to verify prescriptions for local, associated pharmacies.

In some embodiments, the local pharmacy systems 104 of the same region or pharmacy network may have the same urgency or business rules for determining an urgency of prescriptions for ordering in a queue. In these examples, the remote verification application 130 is configured to use the same urgency or business rules for ordering prescriptions. In alternative embodiments, at least some local pharmacy systems 104 of the same region or pharmacy network may have different urgency rules. In these examples, the remote verification application 130 is configured to apply the local rules of each pharmacy when ordering prescriptions. However, each separate list is combined together in a single queue. In some instances, the remote verification application 130 may normalize the different rules to determine respective urgency or priority among the prescriptions from different local pharmacies 104.

Additionally or alternatively, the remote verification application 130 may combine the different rules together for a common list of rules. For example, one local pharmacy 104 may have a rule that specifies certain very important individuals are moved to a top of a queue. Other pharmacies of the same network may not have this rule. However, when applied across all the prescriptions of the same network, the remote verification application 130 orders prescriptions such that those of the designated individuals are moved to a top of the queue.

As discussed herein, the local pharmacy application 120 transmits prescriptions 122 to the remote verification server 102 only when a remote verification feature is locally activated. The local pharmacy application 120 may include a user interface toggle that enables a local pharmacist or technician to activate remote verification. When remote verification is enabled, the local pharmacy application 120 may prompt or otherwise require a local user to record images of the pill preparation process for remote verification. When remote activation is disabled, and verification occurs locally, the local pharmacy application 120 may disable the requirement for images to be recorded (except for preparation of controlled substances). When the local pharmacy application 120 is set to remote verification, the application 120 may transmit a status message to the remote verification system 130, which is used for tracking which local pharmacy systems 104 are in a remote verification mode.

When remote verification mode is enabled, the local pharmacy application 120 may transmit subsequently created prescriptions 122 to the remote verification server 102 (until remote verification is terminated). In other instances, activation of the remote verification option causes the local pharmacy application 120 to transmit all (copies of) prescriptions 122 awaiting review in the local memory device 114 to the remote verification server 102. The local pharmacy application 120 may only transmit or make copies for transmission of prescriptions 122 that contain prescription fill information having at least one image. In these instances, the application 120 does not provide to the server 102 prescriptions 122 that do not contain at least one image. Alternatively, the local pharmacy application 120 may provide copies of all the prescriptions 122 or otherwise make them available to the server 102. The remote verification application 130 only stores, to the remote memory device 110, the prescriptions 122 that contain at least one image.

In some embodiments, the local pharmacy application 120 is configured to activate the remote verification option automatically. Such a feature ensures that patient prescriptions are verified in a timely manner, even when a local pharmacist or technician may not have manually activated the feature. In an example, the local pharmacy application 120 activates the remote verification option when a queue of local prescriptions waiting to be reviewed has reached or exceeded a threshold. In some instances, the threshold may be based on a number of pharmacists assigned to or logged into the local pharmacy system 104. The threshold may be set to a value of 2, 5, 10, 20, etc. for each pharmacist or for the local pharmacy system 104 generally.

In an example, the local pharmacy system 104a may have three assigned pharmacists. At the current time, only two of the pharmacists are logged into the local pharmacy application 120a. A remote verification prescription threshold is set to ten prescriptions per pharmacist at the application 120a. When the number of unverified (e.g., not reviewed) prescriptions 122 reaches twenty, the local pharmacy application 120a activates the remote verification feature. As such, the application 120a requires images to be recorded for subsequent prescription preparation so that the prescriptions 122 (e.g., copies or records thereof) can be transmitted to the remote verification server 102. In some embodiments, the application 120 may also determine of the twenty already created prescriptions 122 stored in the local memory device 114a, which ones include images. In these embodiments, the application 120 transmits to the remote verification server 102 a copy or record of the prescriptions 122 that include at least one image for reducing a prescription queue at the local pharmacy system 104a.

In some embodiments, the local pharmacy application 120 of the local pharmacy system 104 may activate the remote verification option based on an urgency of a prescription. For example, patient information or a medication order may specify a date/time for pickup or have an indication as to whether the medication is needed immediately. The application 120 may detect that the specified date/time is approaching, or that an immediate prescription has not been reviewed within thirty minutes of creation. In response to this determination, the application 120 may activate the remote verification option generally, or at least transmit the urgent prescriptions 122 to the remote verification server 102 for review.

The example application 120 enables a pharmacist or technician to disable a remote verification option. Disabling this option causes a status message to be transmitted to the remote verification server 102. In response, the server 102 removes prescriptions from an ordered list (stored in the remote memory device 110) for review. For prescriptions current being reviewed, the server 102 permits the remote verifier to complete the review, and transmits a verification message to the application 120 after the review is complete. For prescriptions in which problems were flagged remotely, the server 102 may retain those prescriptions for review until the problems are resolved.

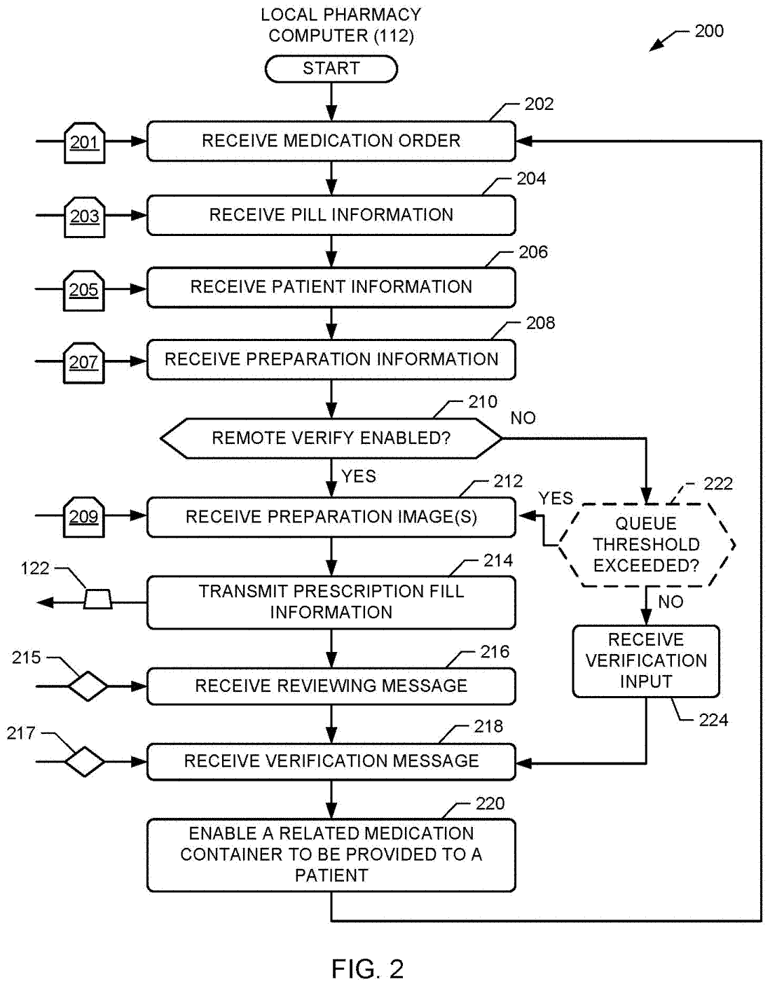

FIG. 2 is a flow diagram of an example procedure 200 for creating and managing prescriptions 122 at the local pharmacy system 104 of FIG. 1, according to an example embodiment of the present disclosure. Although the procedure 200 is described with reference to the flow diagram illustrated in FIG. 2, it should be appreciated that many other methods of performing the steps associated with the procedure 200 may be used. For example, the order of many of the blocks may be changed, certain blocks may be combined with other blocks, and many of the blocks described may be optional. In an embodiment, the number of blocks may be changed based on which data is received in compiling prescription fill information. Further, the step of automatically activating the remote verification option may be omitted. The actions described in the procedure 200 are specified by one or more instructions and may be performed among multiple devices including, for example, the local pharmacy application 120, the local pharmacy computer 112, and/or the local memory device 114.

The example procedure 200 begins when the local pharmacy computer 112 receives a medication order 201 (block 202). The medication order 201 may be received electronically from a clinician system and/or entered by a pharmacist from a paper order. The medication order 201 specifies a patient name, age, birth date, gender, and/or other medications taken. The medication order 201 may also specify a prescriber, a prescriber's DEA number, a medication name, an order identifier, a dosage, and/or a fill quantity. The example local pharmacy computer 112 uses the medication name from the medication order to retrieve pill information 203 from the local memory device 114 (block 204). The pill information 203 comprises standard properties of a medication and includes, for example, a manufacturer name, a dosage form (e.g., oral, inhale, rectal, etc.), a physical shape (e.g., round, oval, square, etc.), a color, a marking, a strength, a DEA code, a lot number, and/or an expiration date. The local pharmacy computer 112 combines the pill information 203 with the medication order 201 to create prescription fill information for the corresponding prescription. In some embodiments, the local pharmacy computer 112 may also receive patient information 205 (block 206). This information may be obtained separately when it is not included with the medication order 201. In some embodiments, the patient information 205 may include an urgency indication, such as a date/time for pickup or indication as to whether the medication is needed immediately. The information 205 may be obtained via a pharmacy form and/or from a patient information database.

The example procedure 200 of FIG. 2 continues as the pharmacist and/or technician prepares the medication. During this process, the local pharmacy computer 112 receives preparation information 207 (block 208). The preparation information 207 includes a count of a number of pills to be dispensed into a medication container for a patient. The pills may be counted by an electronic counter, twice for controlled substances. In some instances, the pills may be weighed by an electronic scale. The count or weight data may be entered into a form provided by the application 120 or received electronically.

The application 120 next determines if the remote verify option is enabled (block 210), and if so, prompts or otherwise requests that one or more images be recorded of the preparation process. The local pharmacy application 120 may request images to be recorded of each side of one pill related to the prescription, the total number of pills in a counting tray, the total number of pills in a medication container, one or more compounding steps, etc. The local pharmacy computer 112 receives one or more images 209 from the camera 118 (block 212). The local pharmacy computer 112 combines the one or more images 209 with the other preparation information 207 as another portion of the prescription fill information, which is stored as a prescription 122 (e.g., an entry or record).

Since remote verify is enabled, the local pharmacy computer 112 transmits the prescription 122, including the corresponding prescription fill information to the remote verification server 102 (block 214). Sometime later, the local pharmacy computer 112 receives a status message 215 from the remote verification server 102 indicative that the prescription 122 is being reviewed (block 216). In response to the message 215, the local pharmacy computer 112 updates the local prescription fill information for the prescription 122 to indicate it is being reviewed. This status update enables a local pharmacist or technician to determine which prescriptions are in process of being verified.

The local pharmacy computer 112 later receives a verification message 217 from the remote verification server 102 (block 218). The message 217 is indicative as to whether the prescription 122 was successfully verified. If the prescription 122 was verified, the local pharmacy computer 112 enables the related medication container to be provided to the patient (block 220). This may include releasing a lock to a compartment 126 of a cabinet 116 or providing a visual indication on the compartment. In other instances, this may include displaying a notification via the local pharmacy application 112 that the prescription 122 is verified. The example procedure 200 then starts again at block 202 for the next medication order.

If the message 217 indicates there is a problem with verification, the application 120 is configured to store to the local memory device 114 one or more reasons for the problem that were identified by the remote reviewer. The one or more reasons may include an incorrect medication entry, an incorrect medication strength entry, an incorrect medication quantity entry, an incorrect directions entry, an incorrect patient data entry, an incorrect medication fill, an incorrect medication strength fill, an incorrect medication quantity fill, a dispenser over-count fill, and/or poor quality of the at least one image. The one or more reasons and indication of the problem are stored in connection with the prescription fill information for the prescription 122. In some instances, the application 120 may display a notification or other visual indicator that is indicative of the unverified prescription. In response, a pharmacist and/or technician of the local pharmacy system 104a may review the reasons and follow established protocols for addressing the problem(s). When finished, the technician and/or pharmacist instructs the local pharmacy application 120 to transmit the updated/corrected prescription fill information, which is routed by the remote verification server 102 to the original reviewer. If the reviewer is no longer available, the server 102 may store the prescription 122 to a front of a list of prescriptions awaiting review. The example process is repeated until the remote reviewer approves the prescription for release of the medication to the patient.

Returning to block 210 of FIG. 2, in some embodiments, the remote verification feature may not be enabled. For example, the local pharmacy computer 112 determines if a queue threshold is enabled and/or has been reached (block 222). If the queue threshold has been reached (or the prescription is deemed urgent), the local pharmacy application 120 activates the remote verification feature and the procedure 200 continues as discussed above for blocks 212 to 220. If the queue threshold has not been reached, the local pharmacy application 120 retains its local verification mode and enables a local pharmacist to provide verification input (block 224). The example procedure 200 then continues as discussed above for blocks 218 and 220.

### II. REMOTE PHARMACY SERVER EMBODIMENT

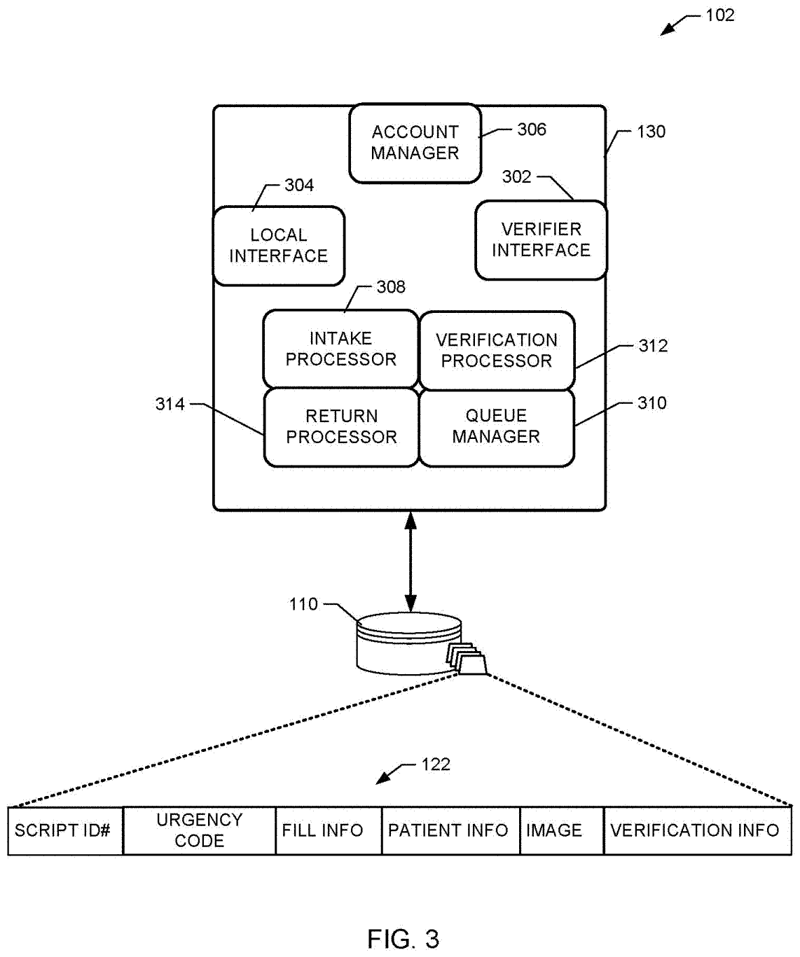

FIG. 3 is a diagram of the remote pharmacy server 102 of FIG. 1, according to an example embodiment of the present disclosure. As discussed above, the remote pharmacy server 102 includes a remote verification application 130 that is configured to manage verification of prescriptions. The illustrated embodiment graphically shows programmed components of the remote verification application 130. Each of the described components may be defined by one or more instructions stored in the memory device. Execution of those instructions by a processor of the server 102 causes the operations to be carried out as described herein.

In some embodiments, the remote verification application 130 is hosted in a Microsoft® Internet Information Service (“IIS”) of the server 102. The IIS may configure the server 102, or at least a portion of the sever 102 including a verifier interface 302, as a webserver. In some embodiments, a local interface 304 may also be configured as a webserver or separate application APIs for operation with the local pharmacy application 120. In some embodiments, the interfaces 302 and 304 may be configured to provide a transport layer security (“TLS”) cryptographic protocol to secure data transmission. Further, in some instances, the interfaces 302 and 304 may be required to exchange security certificates respectively with the local pharmacy systems 104 and/or the remote verifier devices 108. Additionally or alternatively, the interfaces 302 and 304 may provide a virtual private network for connection respectively with the local pharmacy systems 104 and/or the remote verifier devices 108.

The verifier interface 302 is configured for communicative coupling to the remote verifier devices 108. The verifier interface 302 may provide authentication to ensure only registered reviewers may access the server 102. Further, the verifier interface 302 is configured to manage different web sessions with each of the remote verifier devices 108 to enable prescription fill information to be displayed and reviewed.

The example local interface 304 is configured for communicative coupling to the local pharmacy systems 104. The local interface 304 may provide one or more registration forms to enable the local pharmacy application 120 to securely connect to the server 102. For example, the local interface 304 may be assigned an IP address and/or port number. To connect, the local pharmacy application 120 transmits messages to the IP address and/or port number to access the local interface 304. The application 120 may provide a pharmacy name, pharmacy region/group number, site identifier, version number of the application 120, and/or credential information.

The example remote verification application 130 includes an account manager 306 for registering the local pharmacy systems 104, users of the local pharmacy systems 104, remote verifier devices 108, and/or reviewers of the remote verifier devices 108. The account manager 306 provides one more forms or web forms to enable entry of a user name, username, password, DEA number, pharmacy network/region, etc. Registration with the account manager 306 enables the remote verification application 130 to assign users to the same region/company compartment of the server 102. Registration also provides tracking as to which users have access to and/or have reviewed prescription fill information.

The remote verification application 130 is communicatively to the remote memory device 110 for storing records of prescriptions 122. The remote memory device 110 may include a SQL database that is configured for synchronization with SQL databases of the local memory devices 114. In some embodiments, each instance of the remote verification application 130 may be configured as a Central Management Server (“CMS”) for operation with the local memory devices 114. In these embodiments, the local memory devices 114 may be added as linked servers.

An example intake processor 308 of the remote verification application 130 is configured to access or otherwise obtain prescriptions from the local pharmacy systems 104 when a remote verification feature is enabled. In an embodiment, the local pharmacy application 120 transmits a status message to the intake processor 308, via the local interface 304. The status message is indicative of a remote verification mode being enabled. In response, the intake processor 308 transmits a query request message (e.g., a SQL query) for prescriptions. The local pharmacy application 120, operating with the memory device 114, transmits a response message (e.g., a SQL response) to the query. The response message includes a copy of prescription records (that include images) stored at the local memory device 114.

The intake processor 308 is configured to create a temporary list or table of the prescriptions. A queue manager 310 than applies one or more policies or business rules to the temporary list to arrange or order the prescriptions 122 based on an urgency. In addition, the queue manager 310 may adjust policies for time zone differences. It should be appreciated that the local pharmacy systems 104 may share the same policies or have separate policies.

After ordering the prescriptions, the queue manager 310 may add the prescriptions to a master order list or queue that is stored in the remote memory device 110. The master order list includes prescriptions from other local pharmacy systems 104 that are part of the same group or region. The prescriptions are retained in an order based on urgency. The use of one list ensures that the most urgent prescription among all of the local pharmacy systems 104 is verified first/next, instead of having lists complete against each other for review.

In an example, a policy may define urgency codes, where a first code corresponds to a fill next urgency, a second code corresponds to an immediate urgency, a third code corresponds to a high urgency, a fourth code corresponds to a standard urgency, and a fifth code specifies a fill by date/time. A sixth code may be used, which corresponds to when a fill by date/time is approaching or has elapsed, thereby increasing an urgency of the prescription. The queue manager 310 is configured to list or order the prescriptions based on the determined urgency. Additionally, the queue manager 310 periodically reviews the list to promote prescriptions in which a specified date/time has been reached or is approaching.

FIG. 3 shows an example record of a prescription 122, according to an example embodiment. As discussed above, the prescription includes a unique prescription identifier, an urgency code, prescription preparation or fill information, patient information, and at least one image. The prescription 122 also includes a verifier field to track a status of the review/verification. When a prescription is provided to a remote verifier device 108, the queue manager 310 removes the prescription from the ordered list and adds the prescription to an in-process list. When a local pharmacy system 104 ends remote verification, the intake processor 308 receives a corresponding status message, which causes the queue manager 310 to remove the corresponding prescriptions from the ordered list.

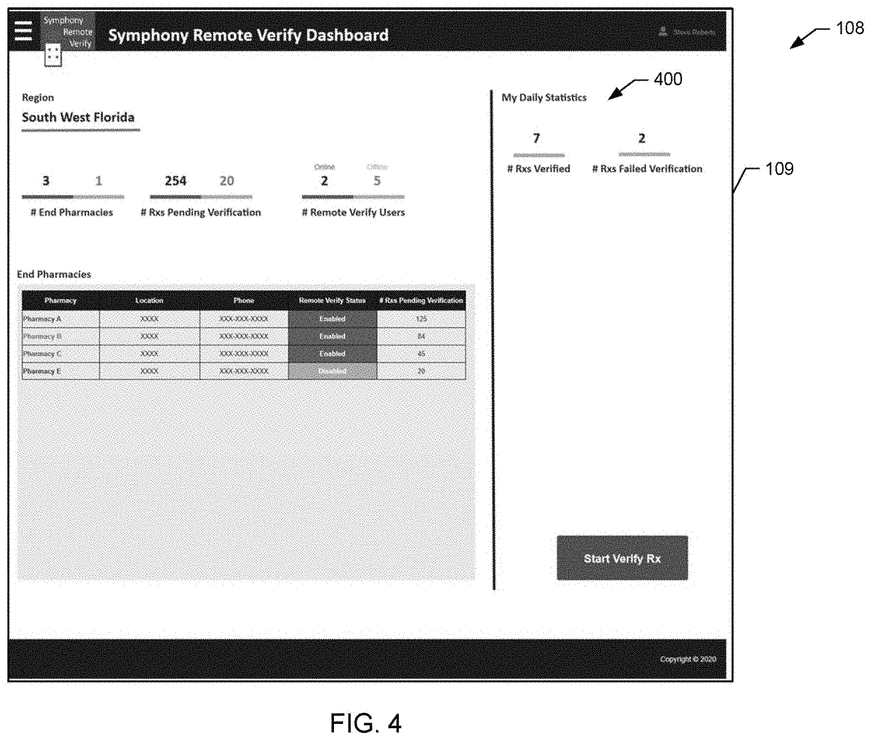

The example remote verification application 130 includes a verification processor 312 that provides for verifications of prescriptions in an ordered list or queue that is provided by the queue manager 310. The verification processor 312 may also transmit or otherwise host one or more dashboards or webpages for the remote verifier devices 108. FIG. 4 is a diagram of an example remote verifier dashboard 400 that is provided by the verification processor 312 of the remote verification server 102 to a remote verifier device 108, according to an example embodiment of the present disclosure. The dashboard 400 is shown as being displayed in a web browsing application 109 of a remote verifier device 108.

The dashboard 400 provides prescription information for a South West Florida region of pharmacies. The dashboard 400 includes information as to which local pharmacy systems 104 currently have remote verification activated. The verification processor 312 may determine this information from status messages received from the pharmacy systems 104.

The dashboard 400 also includes an overview of a number (i.e., 254) of prescriptions awaiting verification on the ordered list and a number of prescriptions (i.e., 20) that are in the process of being verified and included on an in-process list. The dashboard 400 also provides a number of online and offline remote reviewers. In some embodiments, the dashboard 400 also includes personal statistics for the reviewer, such as prescriptions reviewed per day and/or a number of successful/unsuccessful verifications. Selection of a “Start Verify Rx” button on the dashboard 400 causes a request message to be transmitted from the remote verifier device 108 to the verification processor 312.

In response to the request message, the verification processor 312 identifies the most urgent prescription on the ordered list or queue (e.g., the prescription at the top or listed first). The verification processor 312 accesses the corresponding prescription fill information, and enters the information into a template. The verification processor 312 renders the template with the entered information as a webpage having a user interface for remote verification. In addition, the verification processor 312 transmits a status message to the corresponding local pharmacy system 104 that is indicative that the prescription is being reviewed.

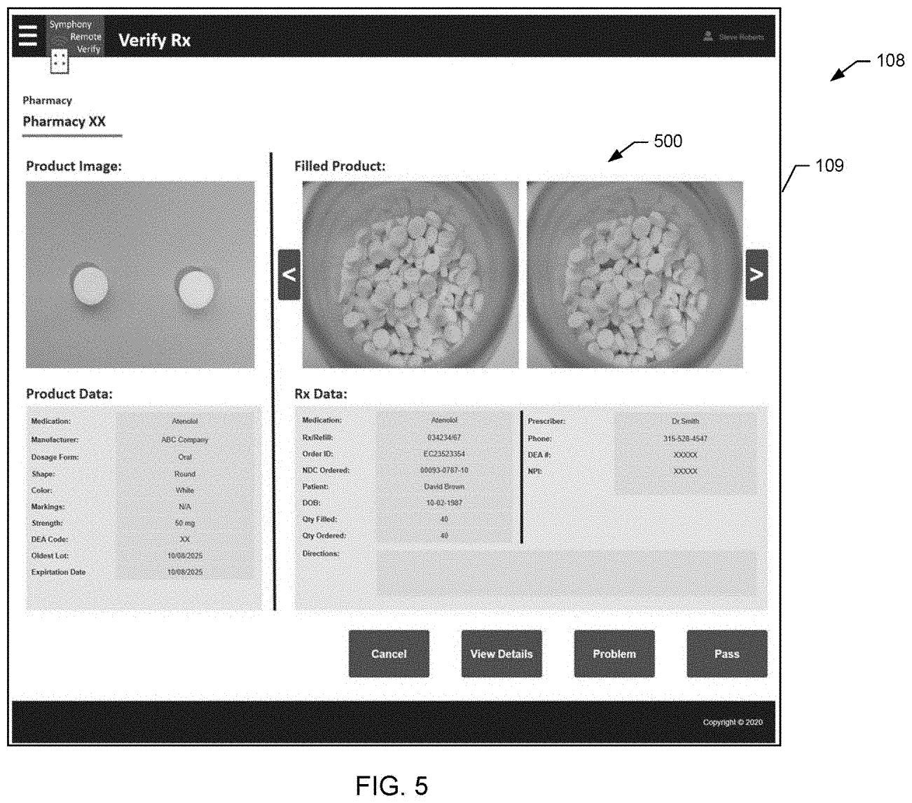

FIG. 5 is a diagram of a verification user interface 500 (e.g., a webpage) provided by the verification processor 312 of the remote verification server 102 to a remote verifier device 108, according to an example embodiment of the present disclosure. The user interface 500 includes a visual display of the prescription fill information of the selected prescription. As shown, this includes images of pill preparation/counting. This also includes a list of medication product data, such as medication name, manufacturer, dosage form, shape, color, marking, strength, DEA code, lot number, and expiration date. The user interface 500 also shows medication order information including the medication name, refill information, order identifier, patient name, patient birth date, a quantity filled, and a quantity ordered. The user interface 500 further provides information regarding the preparer. The user interface 500 enables a feature showing additional prescription fill information via a “View Details” option. Selection of this option causes the user interface 500 to show, for example, a copy of the medication order, information provided by a medication order, additional patient information such as other medications taken, medical history, etc.

The user interface 500 enables a reviewer to verify the prescription by selecting a “Pass” option. Selection of this option causes the web browser 109 on the remote verifier device 108 to transmit a verified message. The verification processor 312 receives and stores the message to the corresponding prescription record/entry. The verification processor 312 also transmits the verified message to the corresponding local pharmacy system 104 so that the respective local prescription entry or record stored in the memory device 114 may be updated. The verification processor 312 may then move the prescription from the in-process list to an approved list. Further, after verification of one prescription, the verification processor 312 may select a current highest listed prescription in the ordered list or queue for display on the remote verifier device 108 for verification of another prescription.

In some embodiments, selection of the “Pass” option causes the verification processor 312 to provide a prompt for the reviewer to scan or take a picture of their a badge with the remote verifier device 108. Information from the scan or the image may be stored to the prescription fill information as verification information, which may be used for auditing pharmacist verification. In some instances, the verification processor 312 compares the scanned/imaged information to registration information to ensure the user is authorized to verify the prescription.

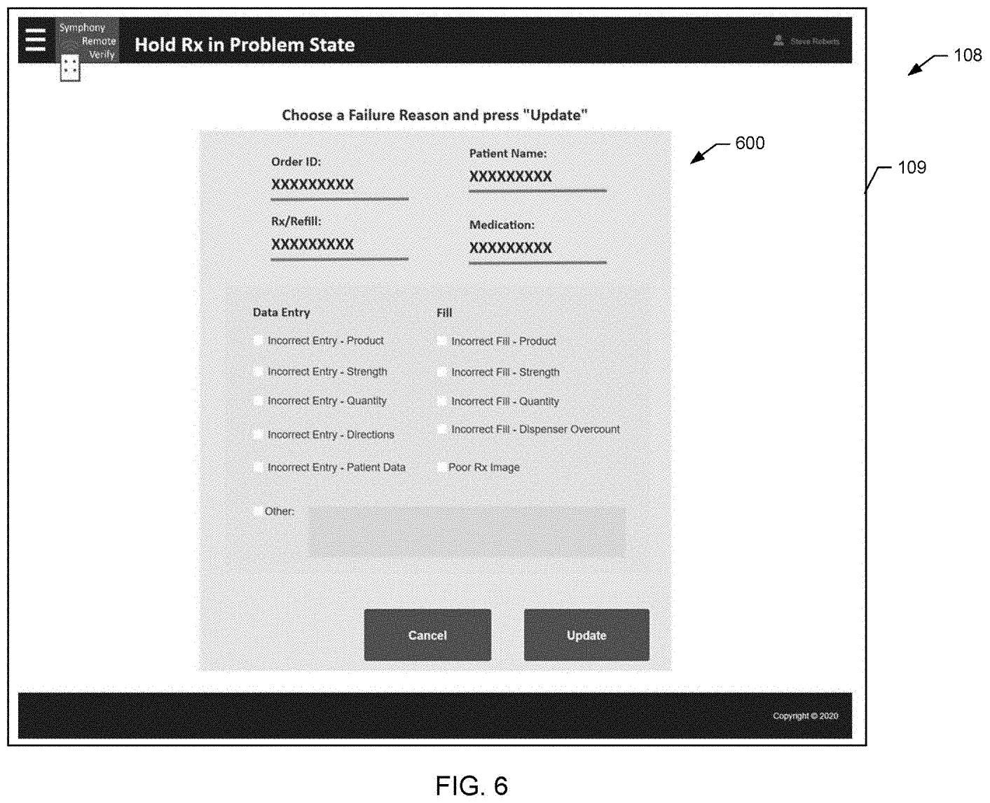

Selection of the “Problem” option in the user interface 500 causes the verification processor 312 to display user interface 600. FIG. 6 is a diagram of a problem user interface 600 (e.g., a webpage) provided by the verification processor 312 of the remote verification server 102 to a remote verifier device 108, according to an example embodiment of the present disclosure. The problem user interface 600 provides selectable options that enable a reviewer to specify reasons that at least some of the prescription fill information appears incorrect. In the illustrated example, the options include an incorrect entry of a medication product, an incorrect entry of a medication strength, an incorrect entry of a pill quantity, an incorrect entry of medication directions, an incorrect entry of patient data, an incorrect fill of a medication product, an incorrect fill of a medication strength, an incorrect fill of a medication pill quantity, an incorrect fill related to a dispenser over-count, and poor image quality. The user interface 600 also provides an option for the reviewer to provide another reason.

Selection of the “Update” option on the user interface 600 causes an unverified message to be transmitted from the verifier device 108 to the verification processor 312. In response to the message, the verification processor 312 moves the prescription from the in-process list to a problem list. In addition, a return processor 314 transmits a notification message to the corresponding local pharmacy system 104 that includes information from the unverified message, including the reasons provided by the reviewer. After a correction is made, the return processor 314 receives a message with the updated/corrected prescription fill information. The return processor 314 adds this information to the prescription entry/record that is in the problem list. The return processor 314 may also transmit a correction message to the remote verifier device 108 indicative that a correction has been made. In some instances, the return processor 314 causes the message or a notification to be displayed on the dashboard 400. The reviewer may select the notification, which causes the user interface 500 to be displayed with the corrected information. The process is repeated via the verification processor 312 and the return processor 314 until the prescription is verified.

It should be appreciated that the dashboard 400 and/or the user interfaces 500 and 600 may be provided by the verification processor 312 in a JavaScript Object Notation (“JSON”), format, a HyperText Markup Language (“HTML”) format, an Extensible Markup Language (“XML”) format, a comma-separated values (“CSV”) format, a text format, and/or a Health-Level-7 (“HL7”) format. In some embodiments, the verification processor 312 converts the prescription fill information into the designated format for display at the remote verifier device 108.

### III. REMOTE VERIFICATION PROCEDURE EMBODIMENT

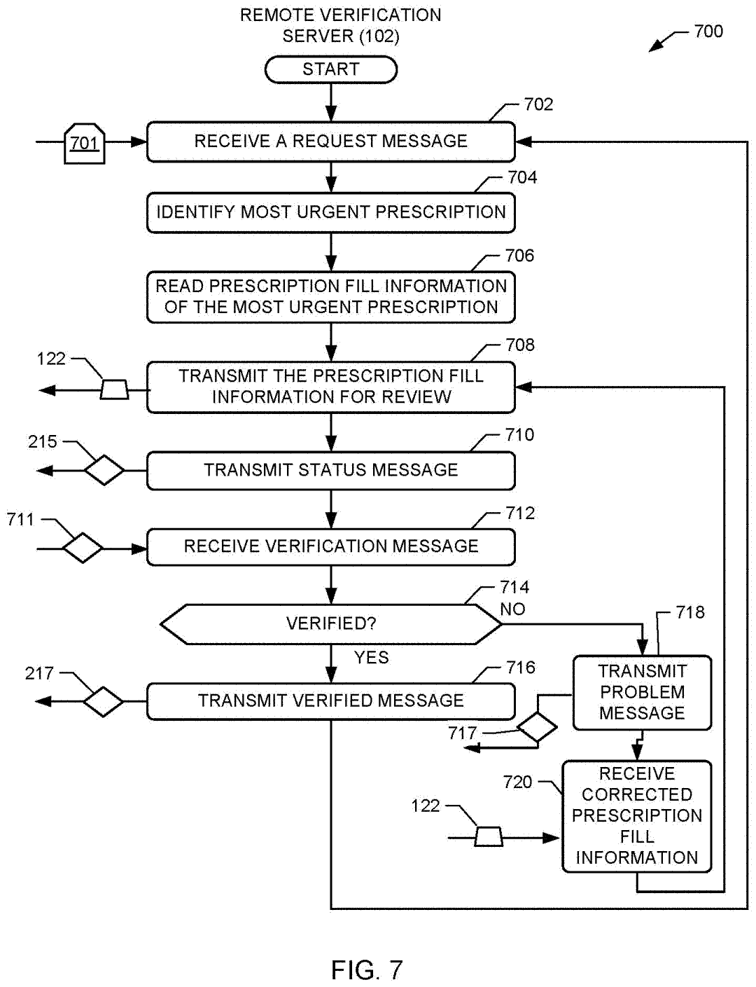

FIG. 7 is a flow diagram of an example procedure 700 for remotely verifying a prescription using the remote verification server 102 of FIGS. 1 to 3, according to an example embodiment of the present disclosure. Although the procedure 700 is described with reference to the flow diagram illustrated in FIG. 7, it should be appreciated that many other methods of performing the steps associated with the procedure 700 may be used. For example, the order of many of the blocks may be changed, certain blocks may be combined with other blocks, and many of the blocks described may be optional. In an embodiment, the number of blocks may be changed based on a process flow for verification. The actions described in the procedure 700 are specified by one or more instructions and may be performed among multiple devices including, for example the remote verification application 130, the remote verification server 102, and/or the remote memory device database 110.

The procedure begins 700 when a request message 701 is received from a remote verifier device 108 to verify a prescription (block 702). In response to the request message 701, the remote verification server 102 identifies a most urgent prescription that is provided in an ordered list of prescriptions (block 704). The most urgent prescription may be sequentially listed first. Alternatively, the most urgent prescription may have a lowest value urgency code value and an earliest creation date among prescriptions with the same lowest urgency code value.

The remote verification server 102 next reads prescription fill information from the selected prescription 122 and enters the read information into fields of a webpage or template for display at the remote verifier device 108 (block 706). The remote verification server 102 then transmits at least some of the prescription fill information provided in the webpage to the remote verifier device 108 (block 708). The remote verification server 102 also transmits a status message (e.g., a reviewing message 215) to a related local pharmacy system 104 indicative of the prescription that is being reviewed (block 710). In some instances, the status message may include an identifier of the prescription or a medication order identifier.

Later after review, the remote verification server 102 receives a verification message 711 from the remote verifying device 108 (block 712). The remote verification server 102 determines from the message 711 if the prescription has been verified (block 714). If the prescription has been successfully verified, the remote verification server 102 transmits a verified message 217 to the local pharmacy system 104, thereby enabling medication associated with the verified prescription to be released to a patient (block 716). The example procedure 700 then begins at block 702 for the next request message 701 received from the remote verifier device 108 to review another prescription.

Returning to block 714, if the prescription was not successfully verified, the remote verification server 102 transmits a problem or notification message 717 that is indicative of the reason(s) the prescription cannot be verified (block 718). The message 717 is transmitted to the local pharmacy system 104 to prompt a pharmacist or technician to correct the one or more identified errors. After correction, the remote verification server 102 receives, from the local pharmacy system 104, an updated prescription 122 with corrected prescription fill information (block 720). The remote verification server 102 then transmits the updated prescription fill information to the remote verifier device 108 for review (block 708). The example blocks 708 to 714, 718, and 720 repeat until a verification message 711 is received that is indicative that the prescription has been successfully verified.

### IV. TWO-STEP VERIFICATION EMBODIMENT

In some embodiments, the example remote verification server 102 discussed above provides for a two-step verification process for prescriptions. A first step of this two-step process comprises verification of prescription information, including patient information checking, patient-medication compatibility checking, dosage checking, and other checking up until a prescription is filled. A second step of the two-step process comprises the checking of the prescription fill information including review of one or more images of a medication preparation process (if available). Either or both of the steps may be performed remotely or locally. For example, the first verification step may be performed remotely, while the second verification step is performed locally where a pharmacist or technician can manually inspect the filled prescription. In some instances, prescription filling cannot begin until the first verification step has been completed.

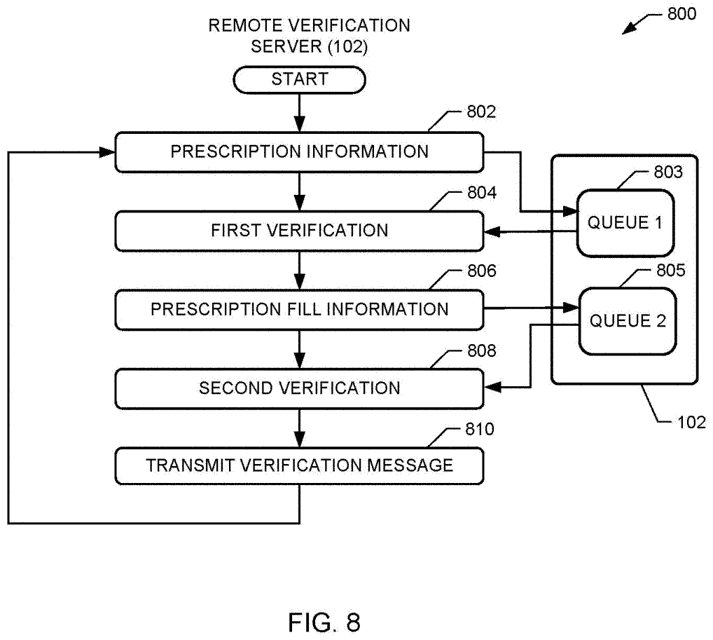

FIG. 8 is a flow diagram of an example procedure 800 for remotely verifying a prescription via the two-step verification process using the remote verification server 102 of FIGS. 1 and 3, according to an example embodiment of the present disclosure. Although the procedure 800 is described with reference to the flow diagram illustrated in FIG. 8, it should be appreciated that many other methods of performing the steps associated with the procedure 800 may be used. For example, the order of many of the blocks may be changed, certain blocks may be combined with other blocks, and many of the blocks described may be optional. In an embodiment, the number of blocks may be changed based on a process flow for verification. The actions described in the procedure 800 are specified by one or more instructions and may be performed among multiple devices including, for example the remote verification application 130, the remote verification server 102, and/or the remote memory device database 110.

The example procedure 800 begins when prescription information is entered into a local pharmacy processor 112 (block 802). The prescription information is generated based on a medication order provided by a clinician. The prescription information includes, for example, a patient name, a patient age, a patient gender, a patient medical condition, a listing of other known medications being consumed by the patient, an identification of a dosage and name of a prescribed medication, a form type of the medication, an urgency to fill the prescription, etc. In the illustrated embodiment, the prescription information is provided to the remote verification server 102, which places the prescription in a first queue 803 based on the corresponding urgency. The first queue 803 includes a list of prescriptions needing first-step verification.

As discussed above in connection with FIG. 7, the remote verification server 102 provides the prescription to a remote verifier device 108 when the prescription reaches a top of the queue. In the illustrated example, the remote verifier device 108 provides a first verification (block 804). During this first verification step, the reviewer may ensure the patient information and medication information matches information in the corresponding medication order. The reviewer also performs a medication-compatibility check for the patient based on other known taken medications and/or known medical conditions of the patient.

After the first-step of the verification process is complete, the local pharmacy 104 may fill the prescription (block 806). As discussed above, this includes counting a specified number of pills/tablets, placing the pills/tablets in a medication container, and printing a label/instructions for the medication container. If remote verification is desired, one or more images of this process are also recorded. In the illustrated example, with remote verification being enabled, the local pharmacy processor 112 transmits the prescription fill information to the remote verification server 102, where it is stored in a second queue 805. When the prescription fill information reaches a top of a queue, it is transmitted by the remote verification server 102 to a verifier device 108, which may or may not be the same device that provided the first-step verification. During this second-step of verification, a reviewer analyzes the one or more images to confirm the pill/tablet color, shape, quantity, weight, etc. is correct and/or the information printed on the label is correct. If the information is correct, the remote verifier device 108 provides a second verification to the remote verification server 102 (block 808). The server 102 then transmits a verification message to the local pharmacy processor 112, which enables the medication associated with the verified prescription to be released to a patient (block 810).

For the above-discussed two-step verification process, the remote verification server 102 provides a dashboard that shows each of the two queues 803 and 805. The server 102 may provide a recommendation or other graphical indication if one queue is larger than the other such that reviewers are directed to attend to the longer queue. In some instances, the remote verification server 102 may only enable one queue to be selected if it is significantly longer than the other queue.

As provided above, the remote verification server 102 enables prescriptions to be quickly verified by a network of remote pharmacists. This configuration prevents local pharmacies from being overwhelmed. At the same time, the remote verification server 102 enables patients to receive prescriptions in a timely manner instead of having to wait for their prescription to be verified. The example remote verification server 102 may enable creation of more remote pharmacies, where a local pharmacist may not be consistently available, thereby improving healthcare coverage to underserved areas. Altogether, the remote verification server 102 discussed herein improves patient healthcare quality in an environment of increased healthcare cost reductions.

### V. CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A pharmacy remote verification system comprising:
a local pharmacy database configured to store prescription fill information for a plurality of prescriptions;
a local pharmacy processor communicatively coupled to the local pharmacy database and configured to receive the prescription fill information for storage in the local pharmacy database, the local pharmacy processor being configured to transmit a set of the prescription fill information for remote verification contingent upon (i) receiving an indication of a remote verification mode being enabled, and (ii) the set of the prescription fill information including images of a prescription preparation process;
a remote pharmacy database configured to store the set of the prescription fill information;
a remote verification server communicatively coupled to the remote pharmacy database and communicatively coupled to the local pharmacy processor via a network connection, the remote verification server including at least one interface configured to provide a remote verifier device access for verification of the set of the prescription fill information, the remote verification server configured to:
arrange prescriptions of the set of the prescription fill information based on an urgency of the prescriptions such that a most urgent prescription is selected for review,
after receiving a request to review a prescription preparation, cause the most urgent, un-reviewed prescription including the corresponding prescription fill information to be displayed on the remote verifier device,
receive a verified message or an unverified message from the remote verifier device,
if the verified message is received, store a verified indication to the prescription fill information of the verified prescription and transmit a verified message to the local pharmacy processor causing the local pharmacy processor to indicate the respective prescription is verified and available to convey to an associated patient, and
if the unverified message is received, store an unverified indication to the prescription fill information of the unverified prescription and transmit an unverified message to the local pharmacy processor causing the local pharmacy processor to indicate at least one reason why the prescription cannot be verified, preventing the respective prescription from being made available to the associated patient, and enabling the at least one reason to be corrected.

2. The apparatus of claim 1, wherein the prescription fill information includes at least one of a patient identifier, a medication identifier, a medication quantity identifier, a dosage identifier, an urgency identifier, a weight value, a medication shape identifier, a medication manufacturer indicator, a dosage form indicator, a color indicator, a marking indicator, a DEA code, an expiration date, medication directions, or at least one image of the prescription preparation process.

3. The apparatus of claim 2, wherein the at least one image includes an image of medication in a medication container, an image of a medication container label, an image of a pill counter display, an image of a weight scale display, or an image of the medication showing a dosage or a name of the medication.

4. The apparatus of claim 1, wherein the at least one reason why the prescription cannot be verified includes at least one of an incorrect medication entry, an incorrect medication strength entry, an incorrect medication quantity entry, an incorrect directions entry, an incorrect patient data entry, an incorrect medication fill, an incorrect medication strength fill, an incorrect medication quantity fill, a dispenser over-count fill, or poor quality of at least one image.

5. The apparatus of claim 1, wherein the remote verification server is configured to transmit a reviewing message to the local pharmacy processor when the prescription fill information of the most urgent prescription related the local pharmacy processor is provided for verification to the remote verifier device.

6. The apparatus of claim 1, wherein the local pharmacy processor is configured to transmit the set of the prescription fill information to the remote verification server if a number of prescriptions needing verification stored in the local pharmacy database exceeds a threshold.

7. The apparatus of claim 1, wherein the urgency includes at least one of a fill next indicator, an urgent importance indicator, a high importance indicator, a standard importance indicator, or a fill by date/time indicator.

8. The apparatus of claim 7, wherein the remote verification server is configured to:
determine a current date/time equals or is about to approach the date/time indicator of an unverified prescription of the set of the prescription fill information; and
change the urgency of the determined unverified prescription to at least one of the fill next indicator or the urgent importance indicator.

9. The apparatus of claim 1, wherein the local pharmacy processor is configured to:
receive an indication that the remote verification mode is terminated; and
transmit a termination message that is indicative of the remote verification mode termination to the remote verification server.

10. The apparatus of claim 9, wherein the remote verification server is configured to receive the termination message and remove, from the remote pharmacy database, prescriptions of the set of the prescription fill information that have not been reviewed.

11. The apparatus of claim 1, wherein the local pharmacy processor is communicatively coupled to the remote verification server via at least one of a private network connection or secure Internet connection, and the remote verification server is communicatively coupled to the remote verifier device via a public network connection.

12. The apparatus of claim 1, wherein the local pharmacy processor is configured to, after receiving the verified message for the respective prescription, cause a medication cabinet to at least one of (i) display a visual indication at a compartment of the cabinet that includes a medication container related to the verified prescription, the visual indication being indicative that the medication container can be conveyed to the patient, or (ii) unlock the compartment of the cabinet that includes the medical container for the verified prescription.

13. A pharmacy remote verification apparatus storing non-transitory computer-readable instructions, which when executed, cause the apparatus to:
receive, from a local pharmacy processor via a first network connection, a set of prescription fill information for remote verification for a plurality of prescriptions;
create an ordered list of prescriptions of the set of the prescription fill information based on an urgency of the prescriptions such that a most urgent prescription is ordered first for review;
receive, from a remote verifier device via a second network connection, a request message to review a prescription;
after receiving the request message, cause the first ordered prescription including the related prescription fill information to be displayed by the remote verifier device;
receive, from the remote verifier device, a verified message or an unverified message;
if the verified message is received, store a verified indication to the prescription fill information of the verified prescription and transmit a verified message to the local pharmacy processor causing the local pharmacy processor to indicate the respective prescription is verified and available to convey to an associated patient; and
if the unverified message is received, store an unverified indication to the prescription fill information of the unverified prescription and transmit a notification message to the local pharmacy processor causing the local pharmacy processor to indicate at least one reason why the respective prescription cannot be verified.

14. The pharmacy remote verification apparatus of claim 13, further comprising stored non-transitory computer-readable instructions, which when executed, cause the apparatus to:
receive, from the local pharmacy processor, a correction message that is in response to the notification message;
transmit at least some information from the correction message to the remote verifier device in relation to the unverified prescription; and
receive, from the remote verifier device, the verified message or another unverified message.

15. The pharmacy remote verification apparatus of claim 13, further comprising stored non-transitory computer-readable instructions, which when executed, cause the apparatus to:
remove the first ordered prescription from the ordered list; and
transmit, to the local pharmacy processor, a verifying message indicative that the first ordered prescription is under review.

16. The pharmacy remote verification apparatus of claim 13, wherein the request message is received from a web browser of the remote verifier device, and
wherein the first ordered prescription, including the related prescription fill information, is formatted for display in the web browser of the remote verifier device.

17. The pharmacy remote verification apparatus of claim 13, further comprising stored non-transitory computer-readable instructions, which when executed, cause the apparatus to:
receive a connection request message from the remote verifier device;
transmit, to the remote verifier device, authentication prompt information;
receive, from the remote verifier device, authentication information; and
contingent upon the authentication information matching at least some authentication information stored in a database, transmit dashboard information that is indicative of a number of prescriptions that are included in within the ordered list of prescriptions.

18. The pharmacy remote verification apparatus of claim 17, wherein the dashboard information includes a total number of connected local pharmacies, an indication of which of the connected local pharmacies have remote verification enabled, and a total number of prescriptions reviewed by a user of the remote verifier device.

19. The pharmacy remote verification apparatus of claim 13, further comprising stored non-transitory computer-readable instructions, which when executed, cause the apparatus to:
determine at least one prescription of the set of prescription fill information does not include at least one image; and
prevent the determined at least one prescription from being included in the ordered list of prescriptions.

20. The pharmacy remote verification apparatus of claim 13, wherein the set of prescription fill information for the plurality of prescriptions is a first set of prescription fill information for a first plurality of prescriptions, and the local pharmacy processor is a first local pharmacy processor,
further comprising stored non-transitory computer-readable instructions, which when executed, cause the apparatus to:
receive, from a second local pharmacy processor via a third network connection, a second set of prescription fill information for remote verification for a second plurality of prescriptions; and
create the ordered list of prescriptions for the first and second sets of the prescription fill information based on the urgency of the prescriptions such that the most urgent prescription is ordered first for review.
